(19) United States
(12) Patent Application Publication
(10) Pub. No.: US 2018/0325723 A1
(43) Pub. Date: Nov. 15, 2018

(54) Temperature and Visibility Regulated Therapy Device

(71) Applicant: Lih-Bin SHIH, San Diego, CA (US)

(72) Inventor: Lih-Bin SHIH, San Diego, CA (US)

(21) Appl. No.: 15/775,908

(22) PCT Filed: Dec. 4, 2015
(86) PCT No.: PCT/US2015/063968

§ 371 Date: May 14, 2018

(51) Int. Cl. A61F 7/02

(52) U.S. Cl. CPC A61F 7/02; A61F 2007/0098

(57) ABSTRACT

A topical temperature therapy device that imparts known temperatures as a function of time to the therapy skin surface of a user during the course of therapy. The skin surface can be intact or breached through injury or surgery. The therapy temperature and time profile can be varied by varying the selections of, among several parameters, the formulation of the heat exchange material and the material and dimensions of the heat exchange material container, thus meeting the needs of a wide range of injuries and demographics. The therapy device contains a temperature indicator indicating the temperature of the heat exchange material in real time. The therapy device further provides visibility to the therapy skin surface during the course of the therapy to allow visual inspection of the therapy area as means to improve therapeutic outcomes. The therapy device is flexible and conforms to the anatomy of the therapy area.

### FIELD OF THE INVENTION

This invention relates to a temperature therapy device providing temperature therapy to an affected skin surface of a user. The invention device provides temperature control and regulation as a function of time on the therapy skin surface, and allows full visibility for a user and/or a care-giver to inspect the therapy skin surface during the course of therapy.

### BACKGROUND OF THE INVENTION

Cold and hot therapy have long been used to address various types of injury, pain and discomfort by a wide range of demographics. Typically one device can serve both purposes by pre-conditioning the device in a different way. For example, either storing the therapy device in a chilled device such as a freezer prior to use in cold therapy, or immersing the therapy device in a hot water bath or heating it up in a microwave oven prior to use in hot therapy. More often, the cold therapy is used to address more critical injuries and its improper use can lead to ineffective clinical goals, or even additional injuries such as ice burn. As a result, discussions of this invention aim at the cold therapy application. But the same device and same advantages can be applied to hot therapy by simply pre-conditioning the therapy device differently.

Cold therapy has long been used to treat various types of musculoskeletal injury, sport injury, soft tissue discomfort and pain. Cold therapy is also commonly applied following trauma, burn, surgery or certain types of medical intervention. Cold therapy is sometimes referred to as cryotherapy. The user of the cold therapy can be a person, with or without a medical condition, whether or not under medical care. Cold therapy is accepted as an effective therapy to reduce pain, swelling, edema and inflammation. The therapeutic effect is achieved by, among many mechanisms, vasoconstriction restricting blood flow to the injured local area and by decreasing cellular activities to improve cell survival. Cold therapy, when used correctly, is recognized to promote healing of an injury and lead to better clinical outcome of a medical procedure.

A cold therapy device can be a simple home-made ice pack, a commercial pre-packaged cold pack, or a wrapping device designed to secure the cold pack to a specific anatomical location such as a knee or an elbow. Cold therapy device can also come with a bulky external power supply to provide continuous or scheduled cooling.

The current cold therapy practice, particularly in the home-setting, is that a user would retrieve the cold pack from a household freezer and subjectively decide whether the temperature of the cold pack is tolerable or not to stay on the treated area. If feeling too cold, the user would use one or multiple layers of towel to wrap the cold pack until “it is the right cold temperature”, before applying the cold pack on the injury site. In a professional setting such as a hospital, a clinic or a physical therapy facility, after the cold pack is retrieved from a chilling device such as a designated freezer, the determination whether the cold pack is too cold or not for the treatment area is often done jointly by the patient and the medical professional. This subjective determination is necessary and a safeguard to prevent ice burn. Ice burn, also named cold pack burn, frostbite or other similar terms, is most commonly caused by prolonged contact with a cold pack at too low temperatures. In some cases, ice burn can cause serious permanent damage to tissues, skins and nerves. There are many reported ice burn injuries resulting from improper use of a cold pack.

However, using user's sensory function to judge the tolerable temperatures of a cold pack is not error-proof and has serious drawbacks. For example, a population with impaired, compromised or still under-developed sensory function or verbal skills, the subjective sensory screening of a cold pack prior to application is deficient. This population includes, but not limited to: a medical patient under sedation, a user with medical conditions such as peripheral vascular disease with reduced sensory function, an elderly, an infant or a toddler, and the like. These populations are thus more vulnerable to ice burn. In fact, even a healthy adult with an injury not considered medically severe, such as muscle strains or sprains, can also result in ice burn when applying cold pack therapy incorrectly. The reasons are several including that the low temperature from a cold pack or a cold device, is known to produce analgesic and anesthetic effects by decreasing nerve conduction to reduce pain. Thus, the initial application of the icing temperature dulls the sensory system of a user and, after that, the user loses certain capacity to tell whether the cold therapy is too cold to be tolerable.

In current practices, particularly in home-settings, the duration of the cold pack application to an injury site is arbitrary. This arbitrary practice, coupled with the wide variations in the types of injury and demographics makes the cold pack therapeutic goal arbitrary and unquantified. Furthermore, available medical guidelines do not suggest a specific temperature range or a specific therapy duration that are considered the most beneficial to achieve a certain therapeutic goal. This again is in recognition that no one cold therapy regiment is applicable to all injury types and to all users. In addition to the large variation in the types of injury, the heat exchange process between the therapy skin area of a user and the cold pack is also different depending on the user's demographic and biologic parameters. For example, the user's age, weight, size, Body Mass Index (BMI), health and disease conditions, or the user's anatomical locations. Different anatomical locations of a user have different tolerances for the cold temperature and its duration. For example, a large surface area such as the user's back or thigh, or a small surface area such as the user's finger, or a bony surface, or a surface with fat tissues . . . et al., all have different cold temperature tolerances and different heat exchange mechanisms with the cold pack.

Notwithstanding the wide variabilities in the application conditions, the medical and the professional therapy communities have recommended the cold therapy duration to be best between 15-20 minutes, and rarely recommending to exceed 30 minutes. The recommended duration also reflects the fact that the treated tissues remain cool even after the cold pack is removed. Studies have shown that the cell hunting response of alternating cycles of vasoconstriction and vasodilation is activated after 10 minutes of cold therapy at temperatures less than 49° F., or 9.5° C. The alternating process of restricting blood flow (vasoconstriction) and delivering of blood rich in oxygen & nutrient (vasodilation) is considered a protective mechanism to tissue survival and essential to healing.

Even though the very word “cold” therapy means a lower than body temperature is being delivered to the body, via the skin surface, to perform a therapeutic function. No commercial cold pack carries any temperature specifications, either the temperature of the coolant inside the cold pack or the temperature impacting on the skin surface under a given set of conditions. Some more sophisticated cooling device provide certain temperature controls, but these devices are often bulky or with a large external power supply restricting mobility of the user. These large externally-powered cooling device may specify the temperature it delivers, but many fail to specify the temperatures when it actually impacts the skin. Some do not provide ideal cooling temperatures to impact the injured area in a timely fashion. For example, a study has shown that an externally powered device reaches the lowest skin surface temperature of 15° C. at 20 minutes without compression, and at 12.5° C. at 20 minutes at high compression. In the same study, an ice bag reached the clinically more significant temperature of 9° C. in about 5 minutes. In an acute trauma or injury, the difference between 5 minutes and 20 minutes to reach the cooling temperature, and the difference between 9° C. and 15° C., are critical and can materially affect the clinical outcome of the medical event.

US 20130261712 A1 made mentions of the reduced skin temperature. However, the reduction of the skin temperature is only by >10° C. This skin temperature lowering may not constitute a cold therapy as the typical healthy skin temperature is at about 34° C. and the ambient temperature approximately 22° C. Thus, the lowering the skin surface temperature by 10° C. is 24° C., still at above ambient temperature and not considered “cold”. Indeed, in one example (FIG. 10 in US 20130261712 A1) shows that the average skin temperature is only reduced to the lowest 17° C. after about 10 minutes and maintained at that temperature for the duration of the 60 minutes in that example. In another example (FIG. 11 in US 20130261712 A1) the delivered skin temperature-time profiles reached the lowest point at approximately 20 minutes, and these temperatures, between approximately 14° C. to above 22° C. at 20 minutes, are far higher compared to the control of the ice bag temperature (at approximately 11° C. at 20 minutes). In two example cold packs in FIG. 11 (in US 20130261712 A1), the skin temperatures remain at above 20° C. for the entire duration, i.e. from time 0 to 60 minutes. These skin temperatures are by no means “cold” or considered therapeutic, particularly when compared to the typical ambient temperature range between 20° C.-22° C. and typical healthy skin temperature range 32° C.-34° C.

Furthermore, US 20130261712 A1 teaches that the coolant freezing temperature to be within the range of −5° C. to +5° C., thus utilizing the latent heat of melting to provide the cooling effect, except that the coolant within this range of melting temperatures does not reach the typical skin temperatures required in a cold therapy as shown in the preceding paragraph. In addition, the United States Food and Drug Administration (FDA) recommends that a household freezer temperature to be below 0° F., or −18° C., and that a household refrigerator at below 40° F. (or 4° C.). In other words, the prior art cold pack is necessarily largely frozen solid after retrieving from a household freezer and its flexibility to conform to a therapy site is compromised.

In yet another prior art, U.S. Pat. No. 8,366,759 teaches a coolant gel formulation, when cooled to between −10° C. and −18° C., the coolant formulation would lower the skin temperature of a body to be about 10° C. to 15° C. for at least 20 minutes. However, U.S. Pat. No. 8,366,759 provides no examples on the skin temperature and its time profile. U.S. Pat. No. 8,366,759 further teaches that the claimed gel formulation has a freezing temperature ranging from about −10° C. to about −6° C. and that the coolant gel contains no ice chunks larger than about 2 cm³. This particular formulation is likely to have larger ice chunks or even mostly frozen if a household freezer is set at the FDA recommended temperature of −18° C., thus compromising at least part of its flexibility and conformity. This prior art is also silent on how the cold pack is applied to the skin surface, whether a typical towel or a cloth is used to wrap the cold pack prior to reaching the claim temperature range of 10° C. to 15° C.

Mainly because of the lack of temperature control and regulation in a cold pack, medical guidelines always recommend wrapping an ice bag or a commercial cold pack with a towel, or “always keep a cloth between your skin and the ice pack”, prior to placing the cold pack on the user's skin surface. The towel, or a cloth, does not serve any therapeutic functions, except to compensate for the lack of temperature control and temperature regulation of a cold pack. Lacking cooling temperature regulation or temperature references in a cold pack is a very serious drawback preventing the proper use of a cold pack to achieve the desired therapeutic result.

Furthermore, current commercial temperature therapy device, cold or hot, provides no visibility to the injury site during therapy time. A great majority of the plastic film enclosure of the coolant in a commercial cold pack is made of a non-transparent plastic or rubbery film enclosure. Even if those made of transparent films, the enclosure surface of a cold pack is always saturated with printed words covering the entire product obscuring the coolant content, thus obscuring the treated skin surface. In other words, once the cold pack is applied to the skin, either an intact skin or a breached skin, the skin surface under therapy is no longer visible to a user, or to a care-giver where a care-giver can be a medical professional caring for a medical patient or an adult caring for a child. In many cases, the coolant in a commercial cold pack is deliberately made to contain a dye or some sort of colorant to obscure visibility to the skin under therapy. The common practice of using a towel or a cloth to wrap the cold pack further ensures that the therapy skin is not visible during the course of therapy. For more complex cooling devices such as a knee wrap or an elbow wrap, the device not only totally covers the injury site, the most common color of the wrapping device is made of black plastic, black fabric or black non-woven.

This long-held practice to obscure the injury site is unknown and has many disadvantages. First, the ice burn may be observed if the therapy skin starts to turn red and redness persists. In an even more serious ice burn, the therapy skin area may turn white. The change of skin color again may only be visually detected by the user or by the care-giver, as the nerve conduction and sensory functions of the user, even for a healthy adult, are already compromised by then. When cold therapy is used after trauma, burn, surgery or a medical intervention involving bleeding, the visibility to the injury site is even more critical. For example, through the inspection of the injury site under the cold pack, the medical professional can determine whether bleeding or oozing continues, or whether hematoma starts to develop, and whether medical intervention is required to address these developing events. The lack of visibility to the therapy skin surface serves no purpose and, in fact, can be harmful as it prevents a user or a care-giver from monitoring the injury site and from taking necessary actions to remedy a developing medical event.

In some applications, cold therapy is used in conjunction with a compression force, either applied manually or with another device. At the present time, no marketed devices for compression cold therapy allows visibility to the therapy skin surface during the course of therapy. The visibility to the therapy skin surfaces is even more important in clinically critical applications, such as trauma or surgery, particularly when involving bleeding from a breached major vessel. Visibility on the therapy skin area allows medical professional to judge whether additional intervention is required. In some clinically critical applications, a rigid transparent therapy device which can support a greater compression force for a sustained period of time is highly advantageous to for these critical applications.

The aforementioned deficiencies such as lacking skin temperature regulation and lacking visibility to the therapy skin surface are applicable when using the same cold pack for hot/heat application. Overcoming the above deficiencies provide the user with greater benefit in both cold and hot therapy.

### SUMMARY OF THE INVENTION

The present invention provides for a temperature therapy device overcoming the aforementioned drawbacks and shortcomings. In addition, the present invention provides novel features and benefits to perform the therapeutic function. The invention therapy device is a topical device transporting and delivering a known cooling or warming temperature profile as a function of time to the therapy skin surface of a user. The invention therapy device can be directly applied to the user therapy skin surface and imparts a known temperature lower than the skin surface temperature to perform cold therapy, or a known temperature higher than the skin surface temperature to perform hot therapy. The selection of the therapy temperatures is consistent with current medical guidelines and clinical evidence in these therapies.

This invention provides the selection of the container material (i.e. the packing and enclosure material for a coolant in the case of a cold pack) so that the material provides a soft and supple feel to the therapy skin when in direct contact with the therapy device. Further, the container material thickness is optimized so that the heat exchange mechanism, between the heat exchange material (HEM, or the “coolant” in cold therapy) and the therapy skin area, delivers a known temperature to the skin surface when in direct contact with the therapy device. This invention also provides formulations of the heat exchange material (HEM) to deliver the desired skin temperature when used in conjunction with a particular container material and its thickness. This invention device may be pre-conditioned, in different manners, to provide either cold or warm therapy to a user. For example, this invention reduces the therapy skin temperatures to be between the therapeutic range of 6° C. to 10° C. within the first 5-10 minutes and that there is a gradual warming trend after the recommended 20-30 minutes of cold therapy. This invention therapy device, when in direct contact with the therapy skin surface, maintains the therapy skin surface temperatures to be between 37° C. to 42° C. for at least 30 minutes in the warm therapy. This invention also provides a temperature indicator to indicate the HEM temperature in real time. This invention device may be used in conjunction with a compression force. This invention therapy device provides visibility to the therapy skin area while conforming to the therapy skin area during the entire duration of the therapy.

In order to elaborate and articulate the concept and practice of this invention, cold therapy examples are used in the following discussions. However, it is to be emphasized that the same invention cooling device can also be used for hot therapy when the therapy device is pre-conditioned differently.

Cold therapy has long been accepted as an effective therapy to treat soft tissue pain or musculoskeletal injury. It has also long been used in the hospital following trauma, burn, surgery or certain types of medical intervention. Even though delivering a cold temperature is the central idea of the cold therapy, none of the marketed cold packs provides information on the cooling temperature, nor the cooling temperature as a function of time, not to mention the cooling temperature as it impacts on the therapy skin surface when performing therapy.

Some sophisticated cooling devices are often bulky, and many equipped with an external power supply, and require to immobilize the user. These devices, even though providing reference temperatures that the device intends to deliver, the indicated temperature, if any, are not the temperature actually impacting on the therapy skin surface. This is because the heat exchange process, between the coolant (or the HEM) in the HEM container and the therapy skin surface, relies on the heat to pass through an interface. The interface, i.e. the container surface in contact with the therapy skin surface, is an integral part of the HEM container construction. The interface is part of the controls and regulations of the heat exchange process. The interface can be rigid or flexible depending on the applications. For example, in a typical flexible therapy device, the interface thickness is between 0.2 mm to 2.0 mm, and a typical rigid therapy device, the interface thickness is greater than 0.7 mm. The present invention provides temperature regulation of a cooling device by incorporating and adjusting the parameters, among other parameters, the HEM formulation, the material property of the HEM polymeric container and the thickness of the interface.

All transparent materials are used to construct the therapy device to render visibility to the entire therapy skin surface during the course of the therapy. The transparency requirement includes, but without limitation, the heat exchange material (HEM) and the HEM container material. The transparent materials may be lightly tinted with color for aesthetic purposes. But even with aesthetic tinting, the visible light transmission (VLT) is specified to between 60% and 100%. VLT is defined by the amount of light in the visible spectrum that passes through the therapy device. The visibility to the therapy skin area is important in preventing serious ice burn or, in even more critical applications, monitoring bleeding, oozing or hematoma formation when blood vessels are breached following trauma, burn or surgical interventions. The selected materials for the interface in contact with the therapy skin area is smooth, soft and supple, making the skin contact an easy and pleasant experience in the midst of pain and injury. The selected material for the interface can be sterilized for critical applications. Or in critical applications, a thin transparent aseptic dressing can be placed on the wound prior to applying the cold therapy device on the wound.

In order to retain HEM transparency, particularly in cold therapy right before the device is retrieved from a freezer, the HEM is selected so that its freezing temperature is −18° C. or below. This selection ensures that the HEM, when storing in a household freezer, remains as a liquid and free of ice chunks or ice crystals upon retrieval for cold therapy use. This selection not only ensures transparency, but also ensures a high degree of device flexibility and conformity.

The invention HEM container further invokes a 3-dimensional design with a height dimension, compared to the marketed cold pack made of two flat sheets specified only with width and length. This design allows the control of HEM volume, the greater the height of the HEM container, the greater the volume of the HEM in the HEM container. The greater the volume of the HEM, the longer the therapy duration given the same set of other parameters.

The HEM container is further compartmented. The number of compartments in one coolant container can be one, or as many as deemed necessary for a particular therapy area or a particular therapy goal. Typically, a household use therapy device has 3-4 compartments. The compartments are separated by walls which are pre-fabricated prior to filling the container with HEM. The walls between compartments can be liquid tight and preventing any liquid HEM exchanges between compartments. The walls can also incorporate small channels to allow a controlled amount of HEM exchange between neighboring compartments. The compartmented HEM container in a flexible device also minimizes pooling of liquid HEM to one side of the device during application. The compartmented HEM container allows even greater flexibility of the device to conform to more challenging anatomical sites.

In a temperature therapy device, the temperature, particularly as the device impacts the therapy skin area, is one of the most important parameters. Utilizing the characteristic of device transparency and the HEM fluidity, a corrosion-resistant temperature indicator is placed inside the liquid HEM and sealed in the HEM container. The temperature indicator can float to the top of the 3-dimensional HEM container if its density is lower than the density of the HEM, but floating at the lower part of the HEM fluid if the density is higher. Regardless, the temperature indicator is mobile within the HEM container and swimming in the HEM to indicate the temperature of HEM in real time.

EP1053726B1 teaches an incorporation of a liquid crystal thermometer into a cold/hot pack, by welding the temperature strip to the inside surface of the pouch. The welded thermometer is visible through the opening of a transparent window in an otherwise non-transparent device. This incorporation in fact reads the temperature between the “fluid/gel-like material” (the “coolant”) and its enclosure directly exposed to the ambient temperature. This way of temperature indication is not the same as having a thermometer inside the HEM (the “coolant”) and fully visible from both sides of the therapy device to provide instantaneous real time temperature reading of the HEM. In addition to floating a thermometer inside the HEM liquid of a transparent therapy device, other decals for informational, functional and decorative purposes may also be incorporated to float inside the HEM container.

The transparent material used in constructing the container to encapsulate the HEM can be flexible or rigid. The flexible transparent material is more likely used in a therapy device with a larger surface area, while the rigid device is often smaller in size for a more targeted application, such as to stop bleeding on a puncture site by a hypodermic needle or a catheter. Both the flexible and rigid transparent HEM containers can be used in conjunction with a compression force.

In one aspect of the invention, the therapy device consists of a heat exchange material (HEM) and a container enclosing the HEM. The HEM container is a container made of polymeric material and used to enclose and seal the HEM inside. In one aspect of the invention, the HEM container is flexible. In another aspect of the invention, the HEM container is rigid. The polymeric materials for the coolant container can be a plastic, a thermoplastic, a rubber, an elastomer, a polymer blend or the combination of more than one material.

In one aspect of the invention, the polymeric material for the flexible HEM container can be selected from a variety of polymeric materials including, without limitation, polyester, nylon, polyacrylamide, polycrylonitrile-polyacrylamide, polycarbonate, polystyrene, polyvinylchloride, low-density polyethylene, high density polyethylene, polypropylene, polyurethane, polyvinylchloride, polyvinyl alcohol, ABS, neoprene, nylon, polyethylene terephthalate, polyethylene glycol, poly-vinyl-pyrrolidone and methacrylates, ethylene vinyl acetate, polytetrafluoroethylene, expanded polytetrafluoroethylene, fluorinated polymer, fluorinated elastomer, cellulose, polyolefin, silicon-containing polymer, polysilicone, a mixture of the aforementioned, or the like.

In one aspect of the invention, the polymeric material for the rigid HEM container can be selected from a variety of polymeric materials including, without limitation, polyester, nylon, polyacrylamide, polycrylonitrile-polyacrylamide, polycarbonate, polystyrene, polyvinylchloride, low-density polyethylene, high density polyethylene, polypropylene, polyurethane, polyvinylchloride, polyvinyl alcohol, ABS, neoprene, nylon, polyethylene terephthalate, polyethylene glycol, poly-vinyl-pyrrolidone and methacrylates, ethylene vinyl acetate, polytetrafluoroethylene, expanded polytetrafluoroethylene, fluorinated polymer, fluorinated elastomer, cellulose, polyolefin, silicon-containing polymer, polysilicone, a mixture of the aforementioned, or the like.

In one aspect of the invention, the selected polymeric material is fabricated into a pre-designed and pre-designated shape and size to form a HEM container. The fabricated HEM container containing HEM has the visible light transmission (VLT) between of 60% and 100%.

In one aspect of the invention, the HEM container has a 3-dimensional structure specified by width, length and height. In one aspect of the invention, the width, length and height are determined by the intended application and can be of a wide range of shapes, sizes and the combination thereof.

In one aspect of the invention, the HEM container has a surface contacting the therapy skin surface and this surface is defined as “the interface”. In one aspect of the invention, the thickness of the interface is the same as the thickness in other parts of the HEM container. In another aspect of the invention, the thickness of the interface is different from other parts of the container depending on the heat exchange requirements. The interface thickness for the flexible transparent therapy device is between 0.2 mm and 2.0 mm, and the thickness of other parts of the flexible container can be thicker if preventing heat loss to the ambient is desired. In one aspect of the invention, the interface thickness for the rigid transparent therapy device is greater than 0.7 mm.

Polymeric materials, including engineering polymeric materials, have a wide range of thermal conductivity, ranging from approximately 1.0 W/(m-K) to above 100 W/(m-K), where W/(m-K) is watts per meter Kelvin. In one aspect of the invention, a low thermal conductive polymer is used as the interface material. In another aspect of the invention, a high thermal conductive polymer is used as the interface material. The selection of the material thermal conductivity is made in conjunction with other parameters, including but not limited to, the selection of the interface material, the selection of the interface thickness and the selection of the HEM formulation. The judicious combination of these parameters serves to reach a particular skin surface temperature-time profile to perform a particular therapy.

In one aspect of the invention, the polymeric material for the flexible transparent therapy device is a soft, pliable and supple material. The softness and flexibility is between Durometer Shore A65 and Shore A90. The softness and flexibility can also be described by the convention used in the flexible film industry, that is, between 2S and 7S. In one aspect of the invention, the polymeric flexible film is a polyvinylchloride, a polyurethane, a silicone, and the like. In one aspect of the invention, the polymeric material contains environmental-friendly plasticizers and meet governmental environment and health regulations of certain regions and countries. In one aspect of the invention, the polymeric material contains one or more anti-microbial agents. In one aspect of the invention, the polymeric material contains additives to retain the structural and property integrity at low temperature so that the container does not become brittle and crack at low temperatures. In one aspect of the invention, the polymeric material passes the standard brittleness low temperature plastic sheeting impact test, also termed as cold crack test, at between −50° F. or below. In one aspect of the invention, the polymeric material is subjected to sterilization for hospital critical applications.

In one aspect of the invention, the HEM container is a rigid transparent HEM container meeting the specified VLT 60% to 100% requirement. In one aspect of the invention, the polymeric material is polycarbonate, high-density polyethylene and the like. In one aspect of the invention, the interface of the rigid transparent HEM container has a slight protrusive curve to allow more comfortable contact with the therapy skin surface. In one aspect of the invention, the thickness of the interface of a rigid transparent HEM container is greater than 0.7 mm depending on application needs. In one aspect of the invention, other dimensions of the rigid transparent HEM container can be of any values depending on application needs.

In one aspect of the invention, the heat exchange material (HEM) for the flexible transparent HEM container is liquid, a sludge, a gel, or a gel-like material et al., with certain flow properties. In one aspect of the invention, the HEM is selected so that when in use in a HEM container, the VLT of the therapy device is between 60% and 100%. In one aspect of the invention, the HEM has the freezing temperature at below −18° C. The HEM can also be a solid for the rigid coolant container, provided that the solid in the frozen state meets the 60%-100% VLT requirement.

In one aspect of the invention, the heat exchange material (HEM) is water containing at least one electrolyte, for example, calcium chloride or ammonium nitrate. In another aspect of the invention, the HEM is water containing a water-soluble or a water-dispersible polymer, such as, for example, sodium carboxymethyl cellulose, cellulose ether, guar gum, sodium polyacrylate, polysaccharide, and the like. In another aspect of the invention, the HEM is selected from various heat transfer materials including, but not limited to, water, propylene glycol, ethylene glycol, polyethylene glycol, triethylene glycol, glycerol, hydrocarbon, hydrocarbon blend, aliphatic hydrocarbon, aliphatic hydrocarbon blend, synthetic alkylated aromatics, synthetic organic hydrocarbon, silicone fluid, dimethylpolysiloxane, molten salts, ionic solids, potassium formate, calcium chloride brine solutions, potassium nitrate, sodium nitrate, lithium nitrate, calcium nitrate, ammonium nitrate. The HEM can be a single component, or the mixture of two, or more than two.

In one aspect of the invention, the HEM is selected from a class of Phase Change Material (PCM) capable of maintaining a narrow melting temperature range, within 1° C.-3° C., at the selected temperature range. The PCM is capable of absorbing or releasing relatively large amounts of latent heat at a relatively constant temperature, typically referring to melting from solid to liquid or solidifying from liquid to solid. In another aspect of invention, the PCM is a salt hydrates. The coolant may be a bio-based fat, fatty acid, ester, oil, or the like. Alternatively, the coolant may be a petroleum-based hydrocarbon, synthetic alkane, ester, mineral oil, paraffin, other organic derivative, and the like. By way of example, the melting temperature of the PCM is selected to be between about −30° C. and about −18° C. The selection of this range allows that the advantage of the latent heat upon melting, but the PCM is only partially crystalized in the freezer and still retains visibility in the liquid state. A PCM material can be used singularly or in combination with another HEM material.

In one aspect of the invention, the heat exchange material (HEM) is a water and glycerin mixture with glycerin constituting from 35% to 75% by volume, or a water and propylene glycol mixture with propylene glycol constituting from 35% to 65% by volume. In one aspect of the invention, the HEM is a water and glycerin and propylene glycol mixture. In one aspect of the invention, the said water mixture contains additives.

In one aspect of the invention, additives such as a thickener or a viscosity enhancer may be added to the HEM to achieve the desired viscosity. The addition of thickener may also lower the freezing temperature of the HEM. In one aspect of the invention, a defoamer may be added to the HEM to remove bubbles and foams. In one aspect of the invention, an inhibitor may be added to improve HEM shelf life and stability. In one aspect of the invention, the colorant may be added to enhance product aesthetics. The addition of additives does not compromise the visibility and the freezing temperature requirements.

In one aspect of the invention, the HEM container is a 3-dimensional structure with a height. In one aspect of the invention, the height of the HEM container is a wall and pre-fabricated. The height of the HEM in HEM container is not a result of overfilling HEM into two flat sheets as in the marketed cold pack products. The height is defined between the distance between the top surface of the interface and the bottom surface of the top cover surface. In other words, the height is defined as the distance of the cavity for HEM. In one aspect of the invention, the pre-fabricated height of a flexible transparent therapy device is between 0.3 cm and 3.0 cm for the flexible HEM container.

In one aspect of the invention, the HEM container is one single continuous compartment. In another aspect of the invention, the HEM has two or more than two compartments, each compartment is connected to one or more than one neighboring compartments. The neighboring compartments are separated by walls. In one aspect of the invention, the partitioning wall of the flexible transparent compartment improves the flexibility of the therapy device by its ability to bend at the partition. In one aspect of the invention, the wall is liquid-tight preventing exchange of HEM between compartments. In another aspect of the invention, the wall has built-in channels allowing a controlled amount of liquid exchange between neighboring compartments. In one aspect of the invention, the height of the wall is selected to meet application needs, as the height of the wall affects the volume of the HEM, thus affecting the therapeutic cooling/warming temperature-time profile. In one aspect of the invention, the wall serves to minimize pooling of liquid HEM during application.

In one aspect of invention, a corrosion resistant thermometer can be placed in the liquid HEM inside the HEM container prior to the HEM container compartments are sealed. The thermometer can be viewed from both sides of the therapy device due to the high visibility of the therapy device. In one aspect of the invention, the thermometer can be a reversible liquid crystal thermometer strip indicating temperature range with the appearance or disappearance of a color. In one aspect of the invention, the thermometer can of other designs. In one aspect of the invention, the thermometer is movable in the HEM, as if swimming in the HEM liquid pool. In one aspect of the invention, the thermometer can be made of a material with the density lower than the HEM density and the thermometer can float to the top. In one aspect of the invention, the density of the thermometer material is equal or higher than the HEM density. In that case, the thermometer is floating in the middle or at a lower part of the HEM liquid. In another aspect of the invention, other functional, informational or decorative decals with a wide range of colors and design are placed inside one or more transparent compartments of the HEM container.

In one aspect of the invention, the therapy device can be strapped to the anatomical location of the therapy skin surface area using common straps which can be attached to the therapy device via a removable Velcro and adhesives or other similar designs.

### BRIEF DESCRIPTION OF THE DRAWINGS

The accompanying drawings, which are incorporated herein and form part of the specification, illustrate various embodiments of the present invention and together with the description, further serve to explain the principles of the invention and to enable a person skilled in the pertinent art to make and use the invention. In the drawings, like reference numbers indicate identical or functionally similar elements. A more complete appreciation of the invention and many of the attendant advantages thereof will be readily obtained as the same becomes better understood by reference to the following detailed description when considered in connection with the accompanying drawings, wherein:

FIG. 1 is the cross-sectional view of an exemplary embodiment of a transparent temperature therapy device. FIG. 1A is a flexible transparent therapy device and FIG. 1B a rigid transparent therapy device.

FIG. 2 is an exemplary embodiment of a flexible transparent temperature therapy device applied to the therapy skin surface, with or without a lesion.

FIG. 3 is an exemplary embodiment of a flexible transparent temperature therapy device. FIG. 3A is the two-parts of a HEM container prior to filling with the HEM and sealing.

FIGS. 3B and 3C demonstrate the full range of flexibility of the exemplary embodiment therapy device.

FIG. 4 is an exemplary embodiment of a flexible transparent temperature therapy device fully transparent at low temperatures, as indicated by both an external digital thermometer and a thermometer embedded to float in the HEM compartment. FIG. 4A demonstrates therapy device visibility at a low temperature shortly after removal from a freezer. FIG. 4B demonstrates therapy device visibility at a higher temperature as the therapy device warms up in the ambient condition.

FIG. 5 is an exemplary embodiment of a flexible transparent temperature therapy device at an ambient temperature demonstrating full visibility. FIG. 5A is without floating labels and FIG. 5B with floating labels.

FIG. 6 is a skin surface temperature-time profile upon placing an exemplary flexible transparent therapy device on the skin surface of a healthy adult to perform cold therapy. The curves in FIG. 6 represent different HEM formulations and different chill conditioning for cold therapy.

FIG. 7 is a skin surface temperature-time profile upon placing an exemplary rigid transparent therapy device on the skin surface of a healthy adult to perform cold therapy.

FIG. 8 is a skin surface temperature-time profile upon placing an exemplary flexible transparent therapy device on the skin surface of a healthy adult to perform heat therapy.

### DETAILED DESCRIPTION OF THE DRAWINGS

In the following detailed description, reference is made to the accompanying drawings which form a part hereof and in which is shown by way of illustration specific embodiments in which the invention may be practiced. These embodiments are described in sufficient detail to enable those skilled in the art to practice the invention, and it is to be understood that other embodiments may be utilized and that structural or logical changes may be made without departing from the scope of the present invention. The following detailed description is, therefore, not to be taken in a limiting sense, and the scope of the present invention is defined by the appended claims.

By “therapy device” is meant a device to provide temperature therapy that consists of a “heat exchange material, HEM” and a “HEM container” containing the HEM within.

By “therapy skin surface” is meant the skin surface where the therapy device is applied to perform therapy. The therapy skin surface can be an intact skin surface or a breached skin surface. An intact skin surface can be a skin surface of muscle strains, sprains, discomfort, pain, bruises or the like. A breached skin surface can be a lesion or a laceration resulting from one or more types of trauma, injury, burn, surgery or medical intervention.

By “user” is meant a person, either with a medical condition that requires cold/hot therapy, or without a medical condition using the therapy device just to relieve general pain and improve comfort. A user can be a medical patient under the care of a medical professional or a physical therapist or the like. A user can be a medical patient in a conscious state or a medical patient under sedation. A user can be an infant or a toddler without fully developed sensory function or verbal skills and under the care of an adult. In some cases, a user can be an animal where the temperature therapy device is deemed necessary by its owner.

By “care-giver” is meant a person providing care to the user in therapy. For example, a care-giver can be an adult caring for a child in need of a temperature therapy. For example, a care-giver can be a medical professional in a hospital, clinic or a physical therapy facility providing care to a medical patient.

By “hot therapy” is meant a warmer than the body core temperature which is provided to the skin surface to perform a therapeutic function. The “hot therapy” is also termed “warm temperature” or “warm therapy” because the temperatures in this therapy imparting on the skin surface is not to cause burn to the skins, and some would describe these temperatures as “warm”.

By “Heat Exchange Material, HEM” is meant a heat-transfer material with certain heat capacity and it exchanges heat with a body when the therapy device is in contact with the therapy skin surface. A HEM is enclosed in a HEM container and pre-conditioned according to the therapeutic need. The “HEM” is termed “coolant” when describing a cold therapy device.

By “HEM container” is meant a container that contains HEM and isolates HEM from the environment. The HEM container can be made of a flexible polymeric film. The HEM container can also be made of a rigid polymeric material.

By “interface” is meant the surface of the HEM container that comes in contact with the therapy skin surface of a user. “Interface” is an integral part of the HEM container and serves to perform heat exchange between the HEM and the therapy skin surface during the course of therapy.

By “compartment” is mean that the entire cooling device is divided into various compartments with walls between neighboring compartments.

By “Phase Changing Material, PCM” is meant a material that melts or solidifies at a narrow temperature range and is capable of storing and releasing a large amount of energy upon phase change.

By “Visible Light Transmission, VLT” is meant the amount of light in the visible spectrum that passes through the therapy device.

By “swimming labels” or “floating labels” is meant one or more decals made of plastics or other materials may be placed in the midst of HEM and inside the HEM compartment, and these labels can move around (“swim”) in the HEM. Depending on the density differences between the swimming labels and the HEM, the swimming labels may be floating to the top of the HEM, or in the middle or towards the bottom of the HEM. In all cases, the swimming labels are not fixed and movable within HEM.

By “mm” is meant millimeter, “cm” centimeter, “ml” milliliter, “L” liter, “° C.” temperature in degree Centigrade and “° F.” temperature in degree Fahrenheit.

“By compartment height” is meant the compartment wall height, or the height of the HEM container. In other words, it is the distance between the top surface of the “interface” facing the HEM side and the bottom surface of the top cover of the container.

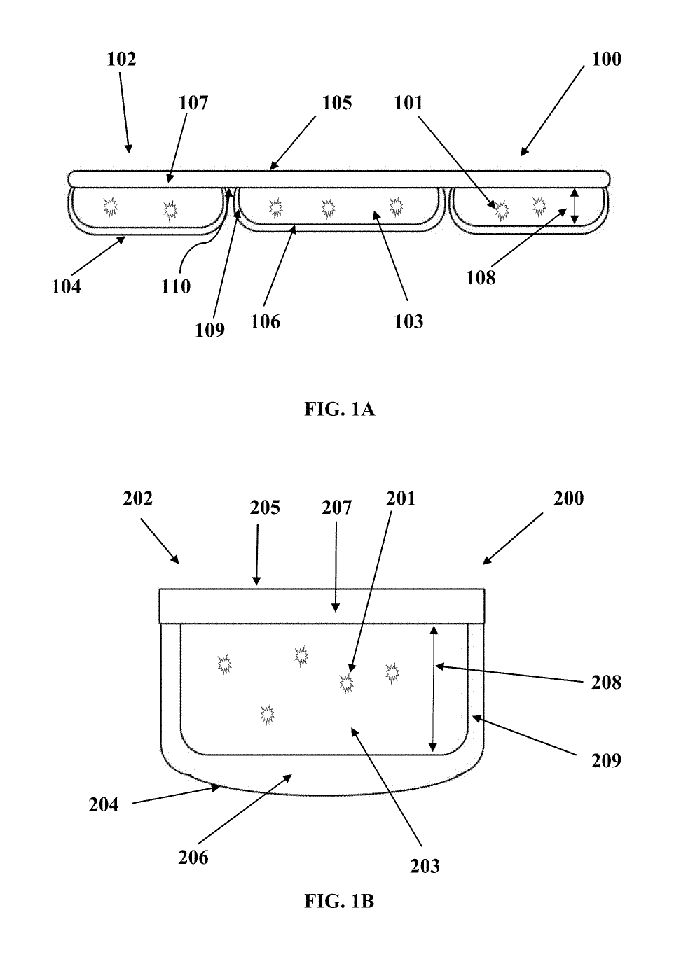

In an exemplary embodiment of FIG. 1A, 100 is the cross-sectional view of a transparent flexible therapy device. The cooling device 100 consists of a heat exchange material HEM 101 and a HEM Container 102. Container 102 has three compartments denoted by 103. Compartment 103 can be fabricated from a flat flexible film by one or more plastic fabrication methods such as vacuum forming, thermal forming, blister forming and the like. The Container has an interface 104 to be in direct contact with the therapy skin surface during therapy and a top cover 105 to seal the Container 102 after the Container 102 is filled with HEM 101. Interface 104 has a thickness of 106 and the top cover 105 has a thickness of 107. Compartment 103 has a height 108. Three compartments are divided by pre-formed partitioning walls 109 and the tip of the walls is sealed to the top surface 105 at the sealing point 110. Similarly, FIG. 1B is an exemplary embodiment of a single compartment transparent rigid therapy device. FIG. 1B has the same numeric notation designations except starting with “2”.

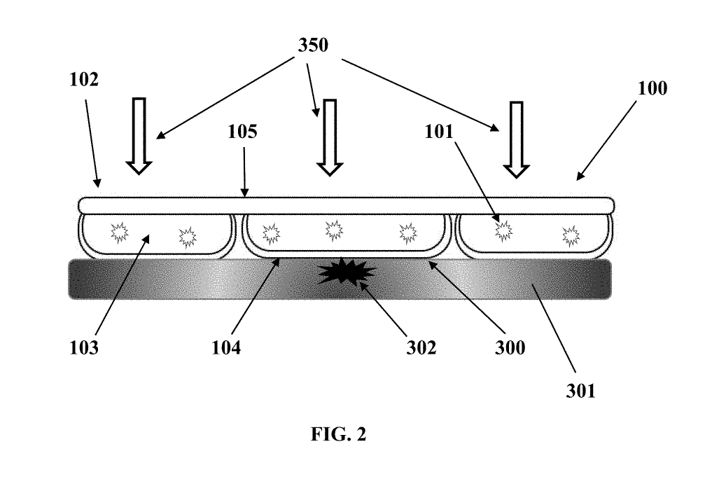

The exemplary embodiments in FIG. 1 can be applied to a therapy skin surface to perform therapeutic function. FIG. 2 is an exemplary embodiment of FIG. 1A as applied directly to the therapy skin surface to perform therapy. The interface 104 of the therapy device 100 is in direct contact with the therapy skin surface 300 of skin layer 301, which may or may not have a lesion 302. FIG. 2 also represents that a compression pressure 350 may be applied on the top surface 105 of the therapy device 100 in the direction substantially vertical to the therapy skin surface 300. Similar inferences can be made when a rigid transparent therapy device 200 in FIG. 1B is applied to a therapy skin surface with or without a lesion, and applied in the presence or absence of a compression pressure.

The materials used in the therapy device in FIG. 1 is a polymeric material. The polymeric material in the fabricated form in container (102 and 202) can be flexible or rigid depending on the fabrication method to meet application requirements. The polymeric materials can be the classes of a plastic, a thermoplastic, an elastomer, a rubber or a polymer blend including but not limited to polyester, nylon, polyacrylamide, polycrylonitrile-polyacrylamide, polycarbonate, polystyrene, polyvinylchloride, low-density polyethylene, high density polyethylene, polypropylene, polyurethane, polyvinylchloride, polyvinyl alcohol, ABS, neoprene, nylon, polyethylene terephthalate, polyethylene glycol, poly-vinyl-pyrrolidone and methacrylates, ethylene vinyl acetate, polytetrafluoroethylene, expanded polytetrafluoroethylene, fluorinated polymer, fluorinated elastomer, cellulose, polyolefin, silicon-containing polymer, polysilicone, a mixture of the aforementioned, or the like.

The polymeric fabrication methods include but not limited to thermal forming, vacuum forming, blister forming, injection molding and the like. For example, the HEM Container 102 in the flexible transparent therapy device 100 in FIG. 1A and FIG. 2 can be fabricated from a pre-formed flexible clear polymeric films with a designated thickness, and the clear flexible polymeric film can be polyurethane or polyvinylchloride and the like. The flexible polymeric film is then thermal-formed or thermal-vacuumed formed into the lower half of Container 102, that is, the Container 102 without the top cover 105. This fabrication process also creates partition walls 109 into 3 compartments 103. After fabrication, liquid HEM is added to fill the compartments. After liquid HEM is filled, top cover is applied to seal the Container. The sealing can be any plastic welding process such as heat seal, radio-frequency seal, sonic welding . . . and the like, or sealed with a chemical adhesive.

Another example is that the HEM Container 202 in the rigid transparent therapy device 200 in FIG. 1B can be fabricated via a conventional injection molding method, and the injection molded parts are sonically sealed to join all parts. After liquid HEM is filled, the filling holes are sealed. Even though the manufacturing operations are different for both the flexible and the rigid therapy devices, their designs are derived from the same concepts and they serve the same functions to provide same benefits.

The dimension, shape and size of exemplary therapy devices in FIGS. 1A and 1B can be as varied as the types of injury and the range of user's demographics, except for the limitations in the Interface thickness (106 in FIG. 1A and 206 in FIG. 2B) as the Interface plays a key role in the heat exchange process between the HEM and the therapy skin surface, and regulates the therapy temperature impacting the therapy skin surface. In the flexible therapy device, the thickness of the Interface 106 is between 0.2 mm to 2.0 mm, and Interface 206 in a rigid therapy device between greater than 0.7 mm. The thicknesses of the top layers 105 and 205 and the walls 109 and 209 can be of any values depending on other design and functional requirements such as preventing heat (or cold) loss to the ambient during therapy time.

The heat exchange material HEM in FIGS. 1A, 1B and 2 can be selected from the materials described in the preceding paragraphs in the section on Summary of the Invention. The HEM is preferably in liquid form taking advantage of its transparency and flexibility. The HEM may contain minor amounts of additives such as a thickening agent, a pH adjusting agent, a defoamer, an inhibitor, a tinting colorant . . . and the like. The additives are to serve various functions. For example, a thickener is to improve the flow properties of the HEM by adjusting its viscosity. The thickener or a rheology modifier can be a natural gum polymer, polyacrylic acid, polyacrylate, a cellulosic or a cellulose derivative such as Carbomer 940™ or Acusol 830™. For example, a defoamer is added to improve the aesthetic appearance of the HEM in a transparent environment by removing bubbles. For example, an inhibitor is added to improve HEM shelf-life and stability. For example, a tinting colorant is added to improve the aesthetic appearance of the therapy device. The addition of the additives does not alter the heat capacity of the HEM and does not compromise the visibility requirement of the therapy device.

The selection criteria for the HEM are several. First, HEM should retain its fluidity at temperatures −18° C. or below. In other words, the selected HEM has a freezing temperature below −18° C. so that when storing in a household freezer, or a similar chilling device in a medical facility, the HEM remains flexible and fluid, for cold therapy use. In the case of a phase change material (PCM) is used, the freezing temperature is elected to be below −18° C.

Another criterion for the selection of HEM is that the selected HEM should have the heat capacity, at a given volume and surface contact area, to generate the cooling and heating temperature-time profile when working in conjunction with the selected Interface material and thickness. Finally, the selected HEM when enclosed in a particular design of a flexible or a rigid Container (102 and 202 in FIGS. 1A and 1B), the combined optical path should be such that the Visible Light Transmission VLT is between 60% and 100% to allow the observation of the therapy skin surface during the course of therapy.

The exemplary embodiments of the therapy device in FIGS. 1A, 1B and 2 may be used in conjunction with a compression force. The exemplary embodiments of the therapy devices in FIGS. 1A, 1B and 2 may be passively powered by pre-conditioning in a household freezer or a chilling device in professional medical facility for cold therapy use. The exemplary embodiments of the therapy devices in FIGS. 1A, 1B and 2 may be passively powered by immersing in the hot water bath or heated in a microwave oven for hot therapy use. The exemplary embodiments of the therapy devices in FIGS. 1A, 1B and 2 may be powered by a battery or by an external AC source to provide cold or hot therapy.

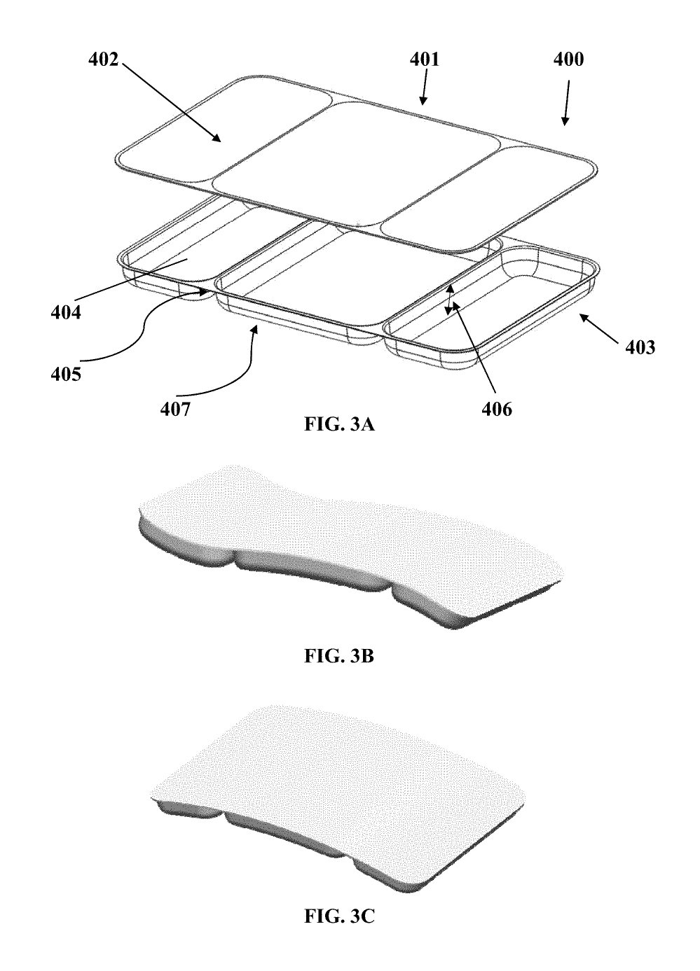

FIG. 3 is a 3-dimensional drawing and rendering of an exemplary embodiment of the flexible transparent therapy device. FIG. 3A demonstrates both parts of the therapy device 400, top part 401 containing top surface 402 and bottom part 403 containing three compartments 404. Therapy device 400 is pre-filled with HEM and pre-sealed Container. The compartment 404 has partition walls 405 and a height of 406. The Interface 407 is the surface on the bottom part 405 which is in contact with the therapy skin surface and not visible in this drawing. FIGS. 3B and 3C are 3-D solid rendering of FIG. 3A demonstrating the full range of flexibility of the exemplary therapy device.

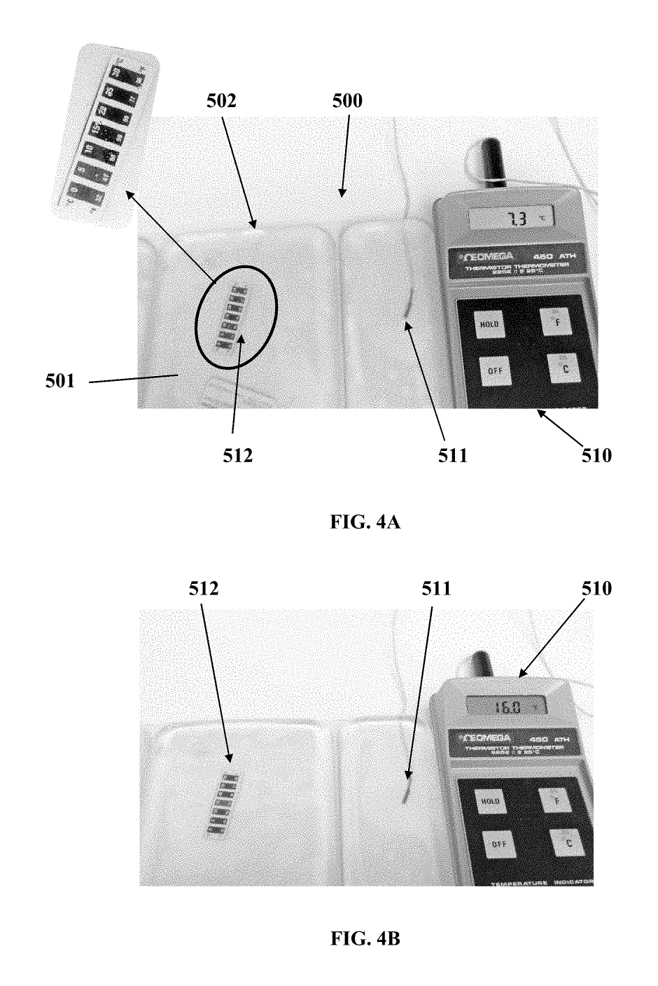

FIG. 4 are photographic representations of an example flexible transparent therapy device 500 at low temperatures. In FIG. 4A, the therapy device 500, consisting of HEM 501 and HEM Container 502, is placed against a flat surface at an ambient condition shortly after taken out of a freezer. A digital thermometer 510 with a small thermocouple probe 511 is placed next to the therapy device 500. The digital thermometer 510 is placed underneath the therapy device 500 against a flat surface. One of the compartment in FIG. 4 contains a color-indicated liquid crystal thermometer strip 512 floating and swimming in liquid HEM 501. The 512 liquid crystal thermometer color indicates between 5° C.-10° C. consistent with the digital thermometer 510 reading of 7.3° C. As the therapy device in FIG. 4A warms up in the ambient conditions, FIG. 4B shows that the 512 liquid crystal thermometer color indicates between 15° C.-20° C., again consistent with the digital thermometer 510 reading of 16.0° C. Both FIGS. 4A and 4B also demonstrate the transparency and visibility of the therapy device. The thermocouple probe 511 under the therapy device and the thermometer 512 inside the therapy device are clearly visible at all times.

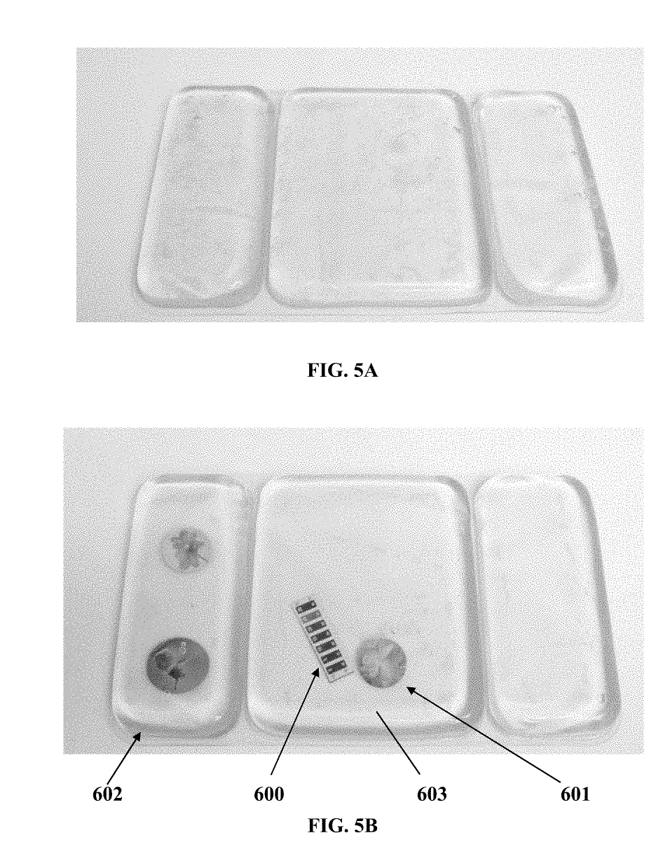

FIG. 5 are photographic representations of an example flexible transparent therapy device at the ambient conditions, again demonstrating transparency and visibility of the therapy device. The therapy device may contain nothing inside as shown in FIG. 5A, or may contain a thermometer 600 and other decorative decals 601 inside HEM Compartment 602 and floating and swimming inside HEM 603. Other informational, functional or decorative decals may be placed in the therapy device in the same manner.

FIGS. 4 and 5 demonstrate a rectangular shaped flexible therapy device with 3 compartments at the approximate width of 15 cm and length 25 cm. It is to be emphasized that the therapy device can be made of any desired shape and size. For example without limitation, rectangular, rectangular with rounded corners, round, oval, oval with different axis ratios, triangular, triangular with rounded corners, hexagon, hexagon with rounded corners, octagon with rounded corners . . . and the like. The shape of the flexible therapy device can also be of an irregular shape, for example without limitation, the shape of an animal, the shape of a flower, the shape of an object . . . and the like. The size of the flexible therapy device can be as small as to provide therapy to a very small surface area, such as a human finger, but can be as large as to provide therapy to the bigger anatomical locations such as the entire back or the thigh of a user. The therapy device can be of one single compartment, two-compartments or more than two compartments. In the multiple compartment design, the compartments can be of equal size and same shape, but also can be of different sizes and different shapes.

The floating labels placed inside the HEM compartment in the midst of HEM as shown in FIG. 5B can serve a particular function or just for decorative purposes. For example without limitation, floating labels can be a thermometer indicating the coolant temperature, a company logo, a user's instruction or a storage information . . . and the like. For example, the swimming labels can be decorative for aesthetic and promotional purposes. For example, a flower, an animal, a cartoon character . . . and the like. The floating labels can be made of a rigid or a flexible polymeric film of a variety of thickness and a wide variety of color and designs. The floating labels can be produced by the silk screen process or by some other similar production methods. The image-printed plastic label can be either use as is, or laminated with thin clear gloss film for protection purposes.

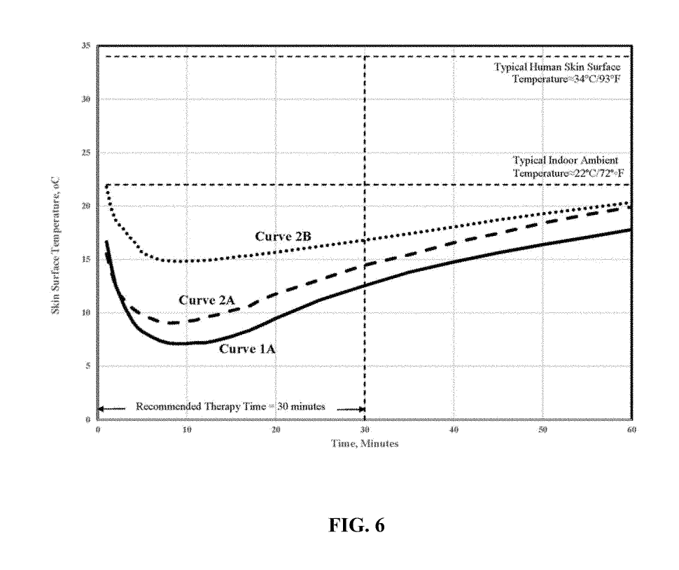

FIG. 6 is an exemplary therapy skin surface temperature of a healthy adult as a function of time upon placement of a pre-cooled transparent flexible therapy device directly on the therapy skin surface. The HEM formulation in Curve 1A is different from that in Curves 2A and 2B, while Curves 2A and 2B are of the same HEM formulation. The difference in the two formulations in FIG. 6 is the different ratios of glycerol and water, and both with a minor amount of additives. The transparent flexible film used in FIG. 6 data is a clear flexible polyvinylchloride at the same thickness of 0.4 mm. The flexible film has the cold crack rating at −40° F. All three curves represent therapy devices with the same interface material and thickness.

Formulation in Curve 1A showed that, at 5 minutes, the therapeutic temperature reaches 8° C. (47° F.) while formulation in Curve 2A reaches 10° C. (50° F.), both in the effective therapy temperature range. The lowest temperatures for Curve 1A and Curve 2A are 7° C. (45° F.) and 9° C. (48° F.) respectively and at approximately 8-10 minutes time. Both formulations maintain at the lowest temperature range for approximately 3-5 minutes before the temperature starts to gradually rise, and rising in a manner consistent with the warming requirement after the cold therapy. At the end of the recommended therapy time of 30 minutes, the therapy skin temperature is warmed up to between 12.5° C. (or 55° F.) and 14.5° C. (or 58° F.), respectively. Skin temperature range between 12.5° C.-15° C. (or 55° F.-58° F.) is considered comfortable cool temperatures, but not the cold temperature that has the potential to cause ice burn. At this time, the therapy device may be removed from the therapy skin surface, or the therapy may continue at warmer temperatures if deemed acceptable by the user or the care-giver. The warming process continues in a gradual gentle manner until reaching the ambient temperature at approximately 1 hour or longer.

Alternatively, the therapy device may be placed in a household refrigerator, instead of a freezer, to produce a gentler and milder cooling temperature-time profile as shown in the formulation in FIG. 6 Curve 2B. The milder and gentler cooling temperatures are particularly suitable for a certain population such as an infant, a toddler, or an elderly and the like

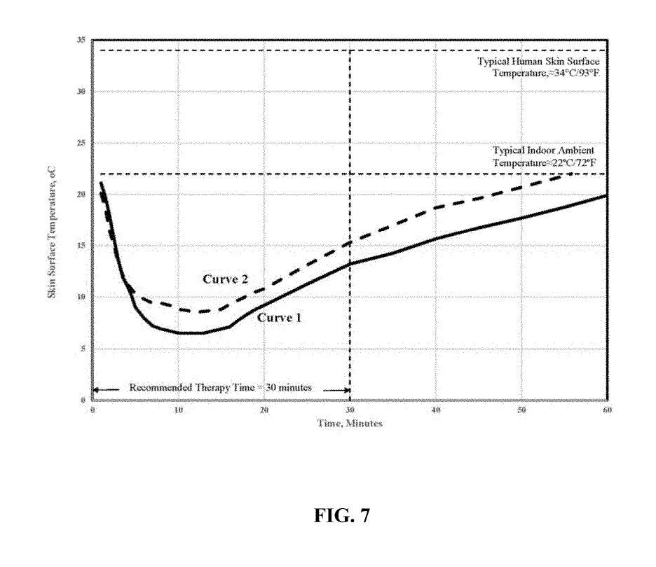

FIG. 7 is an exemplary embodiment of a transparent rigid therapy device. This example, together with examples in FIG. 6, show how the judicious combination of the HEM formulation and the material property and its thickness of the interface affects and regulates the temperature vs. time profile in the course of the cold therapy. Both Curve 1 and Curve 2 in FIG. 7 have the same interface material and thickness, that is, rigid polycarbonate at 0.9 mm thick, much thicker than the interface in the therapy devices in FIG. 6. Curve 1 and Curve 2 in FIG. 7 have different formulations, that is, different ratios of propylene glycol and water. Formulation in Curve 1 showed that, at 5 minutes, the therapeutic temperature reaches 9° C. (or 48° F.) while formulation in Curve 2A reaches 10° C. (50° F.), both are in the effective therapeutic temperature range. The lowest temperatures for Curve 1 at 10 minutes at 7° C. (45° F.) and Curve 2 at 12 minutes at 9° C. (48° F.). Both formulations maintain the lowest temperature plateau for about 10 minutes, again consistent with the known effective therapy temperature range and duration. The combination of the HEM formulation, the interface property and the interface thickness in FIG. 7 also demonstrates that, after the cold therapy, the temperature rises in the warm up cycle in a manner consistent with known recommendations.

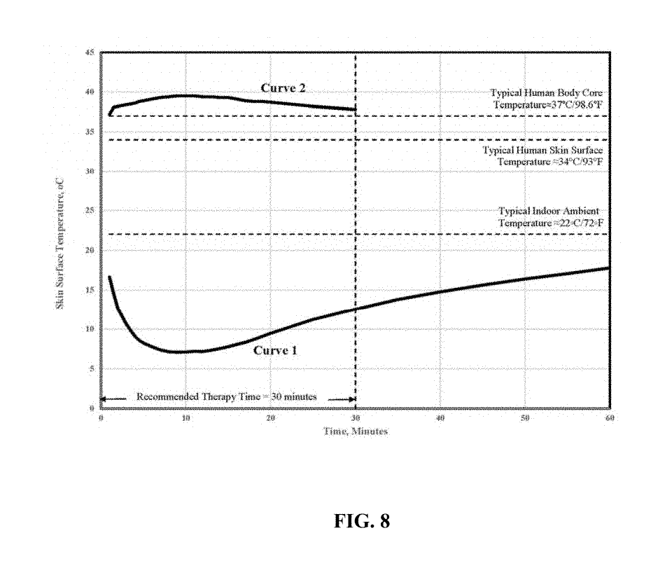

FIG. 8 is the example of applying the therapy device for heat therapy. In FIG. 8, Curve 1 is replicating from Curve 1A in FIG. 6. Curve 2 is the heat therapy temperatures, demonstrating that the same therapy device can be used for both cold and hot therapy. The therapy device in FIG. 7 is heated in a given microwave oven for 80 seconds and the therapy device temperature has a surface temperature determined by a non-contact Infrared thermometer to be 140° F. (or 60° C.). Within 2 minutes of retrieval from the microwave oven, the heated therapy device is placed directly on the therapy skin surface and the therapy skin temperature-time profile is indicated in Curve 2 in FIG. 8. Curve 2 shows that the therapy device imparts a comfortable warm temperature to the therapy skin surface within a narrow range of 38° C. to 39.5° C. of skin surface temperature for the entire duration of 30 minutes. It is to be noted that the therapy device may start with a hot temperature higher than 140° F. (or 60° C.), by simply heating in the microwave oven for additional 15-30 seconds. However, at initial device temperature higher than 140° F. (or 60° C.), it is not recommended to be place on the therapy skin surface directly. The same cautionary practices of using a conventional heating pad applies in this case. Except the invention therapy device provides a temperature reading and allows the direct contact with the skins if pre-conditioning of the therapy device is done correctly.

By way of the following examples, it demonstrates that the invention therapy device can be placed directly on the therapy skin surface to generate controlled, regulated and effective cold or hot temperatures as a function of time to reach the desired therapeutic goals. The invention therapy device further provides transparency and visibility to allow a user or a care-giver to monitor the therapy skin surface in the event an intervention is required. By judiciously selecting and adjusting various parameters, including but not limited to, the HEM formulation, the interface material and its thickness, the therapy device can provide different temperature-time profiles to meet the needs of a wide range of injury types and user demographics.

### Example 1

To prepare a thickened solution of 39% glycerol by volume and 61% water by volume, place 488 ml of DI water in a 1.5 L beaker on a magnetic stirring plate. Add 0.6 grams of liquid triethanolamine into DI to adjust the pH to 10.0 while stirring. The magnetic stirrer in the beaker is removed and 0.8 grams of carbomer 940 in fine powder form is added to the pH-adjusted DI water. Carbomer is a thickener specified as polyvinyl carboxy polymer crosslinked with ethers of pentaerythritol. Separately, 312 ml of neat phase glycerol is measured and placed in another beaker. A hand-held mixer is inserted into the DI and turned to high speed and mix the powdery carbomer into DI water. As the DI is being thickened, which takes about 20-30 seconds, glycerol was poured into the DI slowly while continuing mixing. The mixing continues for another 60 seconds until the mixture appeared to be homogeneous. The mixture solution is let stand at room temperature for about 24 hours until the bubbles settle and disappear. The solution has the viscosity of 50 centipoise and ready to use. Separately, the bottom half of the therapy device 403 as shown in FIG. 3A is formed with a cold crack rated Polyvinylchloride (PVC) Clear Flexible film at 0.4 mm, via thermal vacuum forming process and ready to use. The compartments are filled with the prepared glycerol and water mixture. 225 ml of HEM solution to the center compartment and 125 ml to each of the side compartment. Radio-frequency is applied to seal the bottom part (403) of therapy device cooling device to the flat top 402 film as shown in FIG. 3A. The top film in this example is the same PVC film except 0.8 mm thick, a result of laminating two 0.4 mm films together. The sealed therapy device is placed into a freezer at 2° F. for 24 hour.

After the flexible therapy device is retrieved from the freezer, it is placed on the unbroken skin surface of a healthy adult. The timer starts when the therapy device leaves the freezer and interface side (the 0.4 mm film side) of the therapy device is placed on the skin surface within 30 seconds after retrieval from the freezer. A thermocouple 511 of the digital thermometer 510 in FIG. 4A is placed in the center of the middle compartment of the cooling device (refer to FIG. 3A) and between the therapy skin surface and the interface of the therapy device (refer to FIG. 4A). Temperature recording starts at 1 minute after retrieval from the freezer. At 5 minutes, the digital thermometer reads 9.0° C., at 10 minutes 7.1° C., at 20 minutes 8.7° C., at 30 minutes 11.5° C. and at 60 minutes 16° C.

### Example 2

This example utilizes the same transparent flexible cooling container with the same interface thickness and top film thickness as in EXAMPLE 1. The formulation is different, that is, 60% glycerol by volume and 40% DI water by volume, also with pH adjustment by triethanolamine and thickener Carbomer 940. Temperature recording for this formulation under same testing conditions are as follows: At 5 minutes, the digital thermometer reads 10.9° C., at 10 minutes 9.5° C., at 20 minutes 10.2° C., at 30 minutes 12.3° C. and 60 minutes 17.5° C.

EXAMPLE 3 utilizes a transparent rigid cooling device. The HEM Container has a slightly oval surface at length of 6.2 cm and width 4.0 cm (Refer to its cross-sectional view in FIG. 1B). The height of this coolant container is 3.5 cm. The rigid coolant container is made of polycarbonate material and the interface thickness is 0.9 mm. The HEM for this application is propylene glycol and water mixture at the volume ratio of 30% and 70% respectively. No other additives is added in this example. After 24 hours in a freezer, the therapy device is retrieved and the same measurement conditions specified in EXAMPLE 1 are followed to record temperature and time. The recorded time-temperature profile of the thermocouple between the therapy skin surface and the HEM Container interfaces are as follows: at 5 minutes 9.0° C., at 10 minutes 6.7° C., at 15 minutes 6.7° C., at 20 minutes 9.9° C., at 30 minutes 12.6° C. and finally at 60 minutes 21.2° C.

The foregoing has described the principles, embodiments, and modes of operation of embodiments of the present invention. However, the concept should not be construed as being limited to the particular embodiments described above, as they should be regarded as being illustrative and not as restrictive. Modifications and variations of the disclosed embodiments are possible in light of the above teachings. It is therefore to be understood that the present concept may be practiced otherwise than as specifically described herein. It should be appreciated that variations may be made in those embodiments by those skilled in the art without departing from the scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the concept. It should be understood that various alternatives to the embodiments described herein may be employed in practicing the present concept. It is intended that the following claims define the scope of the invention and that methods and structures within the scope of these claims and their equivalents be covered thereby.

1. A topical temperature therapy device for providing cold and warm temperatures to perform a therapeutic function to a skin surface requiring therapy on a user, comprising:
a heat exchange material; and
a heat exchange material container, and
wherein the heat exchange material container encapsulates and encloses the heat exchange material to form the therapy device, and together have sufficient visible light transmission (VLT) to permit visual inspection of the therapy skin surface, through the therapy device, during the course of therapy.

2. The therapy device according to claim 1, having a VLT value between 60% and 100% for a majority of a surface area of the therapy device throughout the entire device and throughout the duration of a therapy.

3. The therapy device according to claim 1, wherein the heat exchange material is a liquid at room temperature possessing a freezing temperature at −18° C. or below;

4. The therapy device of claim 3, wherein the heat exchange material is water containing at least one or more of the following: an electrolyte, a salt, a water-soluble polymer, or a heat transfer material comprising a class of phase change material (PCM) where PCM is a class of material capable of maintaining a narrow melting temperature range at the selected temperature range.

5. The therapy device of claim 3, wherein the heat exchange material is a water and glycerin mixture with glycerin constituting from 35% to 75% by volume, or a water and propylene glycol mixture with propylene glycol constituting from 35% to 65% by volume.

6. The therapy device of claim 5, wherein the heat exchange material contains an additive such as a rheology modifier, a stabilizer, a defoamer or a colorant.

7. The therapy device of claim 1, wherein the heat exchange material container is made of a clear flexible polymeric material.

8. The therapy device of claim 7, wherein the polymeric material is selected from one of a plastic, a thermoplastic, an elastomer, a rubber, or a polymer blend.

9. The therapy device of claim 7, wherein the flexible heat exchange material container is a clear flexible polyurethane or a clear flexible polyvinylchloride.

10. The therapy device of claim 8, wherein the heat exchange material container comprises a wall member that imparts a height to the therapy device, the wall fabricated prior to the container being filled with the liquid heat exchange material.

11. The therapy device of claim 9, wherein the heat exchange material container comprises a wall member that imparts a height to the therapy device, the wall fabricated prior to the container being filled with the liquid heat exchange material.

12. The therapy device of claim 1, wherein the heat exchange material container is made of a clear rigid polymeric material.

13. The therapy device of claim 12, wherein the rigid heat exchange material container is selected from one of a plastic, a thermoplastic, an elastomer, a rubber, or a polymer blend.

14. The therapy device of claim 7, wherein the surface of the flexible heat exchange material container on the therapy skin side has a thickness between 0.2 mm and 2.0 mm.

15. The therapy device of claim 1, wherein the heat exchange material container comprises one single compartment, or two compartments or more than two compartments, each divided by a wall with the neighboring compartment to allow either no liquid exchange between compartments, or a controlled amount of liquid exchange between compartments.

16. The therapy device of claim 1, wherein the heat exchange material container contains one or more floating labels floating in the midst of the heat exchange material serving functional, informational or decorative purposes.

17. The therapy device of claim 16, wherein the floating label is a temperature indicator indicating temperature of the heat exchange material in real time during the course of cold or warm therapy.

18. The therapy device of claim 1, wherein when in contact with human skin, configured to deliver cold therapeutic temperatures 6° C. to 12° C. to the therapy skin surface within the first 5-10 minutes of contact, maintain a skin surface temperatures between 7° C. to 12° C. for 10-15 minutes before reaching a skin surface temperature range of 12° C. to 15° C. at 30 minutes after contact.

19. The therapy device of claim 1, wherein when in contact with human skin, configured to deliver a warm therapeutic temperature within a range of 37° C. to 42° C. for a duration of 30 minutes.

20. A method of applying the temperature therapy device of claim 1, to a user's skin surface, comprising:
contacting the topical temperature therapy device to the skin surface;
imparting a therapeutic temperature as a function of time to the skin surface under therapy;
viewing the therapy skin surface through the therapy device during the course of therapy, wherein the device has a VLT value between 60% and 100% through a majority of the device;
viewing a temperature indicator inside the therapy device and within a line of sight between the therapy device and the skin.
delivering cold therapeutic temperature of 6° C. to 12° C. to the therapy skin surface within the first 5-10 minutes of contact of the therapy device to the skin; and
maintaining a skin surface temperatures between 7° C. to 12° C. for 10-15 minutes before reaching a skin surface temperature range of 12° C. to 15° C. at 30 minutes after contact.
delivering a warm therapeutic temperature within a range of 37° C. to 42° C. for a duration of 30 minutes, while contacting the skin with the therapy device.

21. A method of constructing a temperature therapy device of claim 1, for providing cold and warm temperatures to perform a therapeutic function to a skin surface requiring therapy on a user, wherein:
providing cold temperatures comprises:
delivering cold therapeutic temperatures of 6° C. to 12° C. to the skin surface within the first 5-10 minutes of contact of the therapy device to the skin; and
maintaining a skin surface temperatures between 7° C. to 12° C. for 10-15 minutes before reaching a skin surface temperature range of 12° C. to 15° C. at 30 minutes after contact;
providing warm temperatures comprises delivering warm therapeutic temperatures within a range of 37° C. to 42° C. for a duration of 30 minutes, while contacting the skin with the therapy device;
the method comprises:
selecting a heat exchange material;
selecting a polymeric material for a heat exchange material container to encapsulate and enclose the heat exchange material to form the therapy device;
selecting a height of the heat exchange material container;
selecting an interface thickness;
wherein the selected heat exchange material and heat exchange material container together have a VLT value between 60% and 100% for a majority of a surface area of the therapy device throughout the entire device and throughout the duration of a therapy;
assembling the heat exchange material and heat exchange material container having a height, with a temperature indicator within the heat exchange material and visible from the outside of the container;
wherein the selected heat exchange material, heat exchange material container, height, and interface thickness together provide either a cold therapeutic temperature or a warm therapeutic temperature as a function of time based on the selected heat exchange material, heat exchange material container, height, and interface thickness.
